(19) United States
(12) Patent Application Publication
(10) Pub. No.: US 2026/0060843 A1
(43) Pub. Date: Mar. 5, 2026

(54) OPTICAL COHERENCE TOMOGRAPHY (OCT) FIBER PROBE FOR GUIDING GLAUCOMA SURGERY

(71) Applicant: Alcon Inc., Fribourg (CH)

(72) Inventors: Corey STEWART, Laguna Niguel, CA (US); Adela APOSTOL, Laguna Hills, CA (US); Kevin Christopher CANZONE, Coto de Caza, CA (US); Mikhail OVCHINNIKOV, Trabuco Canyon, CA (US); Keith Kiyoshi WATANABE, Irvine, CA (US)

(21) Appl. No.: 19/317,512

(22) Filed: Sep. 3, 2025

Related U.S. Application Data
(60) Provisional application No. 63/690,685, filed on Sep. 4, 2024.

(51) Int. Cl. A61F 9/008

(52) U.S. Cl. CPC A61F 9/008; A61F 2009/00851; A61F 2009/00891; A61F 2009/00897

(57) ABSTRACT

A system includes a light source configured to emit treatment light and an optical coherence tomography (OCT) device. The system further includes a surgical instrument including a distal portion configured to be inserted into an eye of a patient. An optical fiber is coupled to the light source and to the OCT device and (a) conducts the treatment light along the distal portion to be emitted from a tip of the distal portion and (b) conducts detection light from the OCT device along the distal portion to be emitted from the tip of the distal portion and conduct a reflected portion of the detection light to the OCT device. A controller obtains image data from the OCT device and operates the light source per the image data, e.g., selects a number of pulses for the treatment light based on a thickness of a trabecular meshwork apparent in the image data.

### CROSS-REFERENCE TO RELATED APPLICATIONS

This application claims benefit of and priority to U.S. Provisional Patent Application No. 63/690,685, filed Sep. 4, 2024, which is hereby incorporated by reference in its entirety as though fully and complete set forth herein.

### INTRODUCTION

Glaucoma, a group of eye diseases affecting the retina and optic nerve, is one of the leading causes of blindness worldwide. Most forms of glaucoma result when the intraocular pressure (IOP) increases to pressures above normal for prolonged periods of time. IOP can increase due to high resistance to the drainage of the aqueous humor relative to its production. Left untreated, an elevated IOP causes irreversible damage to the optic nerve and retinal fibers resulting in a progressive, permanent loss of vision.

Glaucoma is often treated by inserting an instrument through the cornea in order to make an incision or place a shunt or incision in the anterior chamber to facilitate drainage of fluid from the anterior chamber. A shunt may be placed, for example, in the trabecular meshwork, Schlemm's canal, suprachoroidal space, or elsewhere. During the treatment, the surgeon will view the anterior chamber and the instrument through a gonioscope or an ophthalmic microscope in order to place the incision or shunt at an appropriate location with the application of an appropriate amount of pressure.

It would be an advancement in the art to facilitate the performance of effective glaucoma treatments.

### SUMMARY

In certain embodiments, a system includes a light source configured to emit treatment light in order to alter patient tissue and an optical coherence tomography (OCT) device. The system further includes a surgical instrument including a distal portion configured to be inserted into an eye of a patient. The system further includes an optical fiber coupled to the light source and to the OCT device, the optical fiber configured to (a) conduct the treatment light along the distal portion to be emitted from a tip of the distal portion and (b) conduct detection light from the OCT device along the distal portion to be emitted from the tip of the distal portion and conduct a reflected portion of the detection light received at the tip of the distal portion to the OCT device. A controller is coupled to the OCT device and the light source, the controller configured to: obtain image data from the OCT device; and operate the light source according to the image data.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, and may admit to other equally effective embodiments.

FIG. 1 illustrates an example operating environment for providing glaucoma treatments using an illuminated ophthalmic surgical instrument in accordance with certain embodiments.

FIG. 2 is a cross-sectional view of the eye with an inserted illuminated ophthalmic surgical instrument in accordance with certain embodiments.

FIG. 3 is a side view of the ophthalmic surgical instrument of FIG. 2 and an optical coherence tomography (OCT) device for providing guidance during ophthalmic surgery in accordance with certain embodiments.

FIG. 4 is a process flow diagram of an example method for providing guidance during ophthalmic surgery in accordance with certain embodiments.

FIG. 5A illustrates an example probe of the ophthalmic surgical instrument of FIG. 2 in use during a glaucoma treatment in accordance with certain embodiments.

FIG. 5B illustrates an example output of the OCT for the method of FIG. 4 in accordance with certain embodiments.

FIGS. 6A to 6E are cross-sectional views of optical fibers that may be used in accordance with certain embodiments.

FIGS. 7A and 7B illustrate distal ends of optical fibers that may be used in accordance with certain embodiments.

FIG. 8A illustrates a rotating optical fiber that may be used in accordance with certain embodiments

FIG. 8B illustrates image data that may be captured using the rotating optical fiber of FIG. 8A in accordance with certain embodiments.

FIG. 9 illustrates an example computing device that implements, at least partly, one or more functionalities for providing guidance during an ophthalmic surgery in accordance with certain embodiments.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

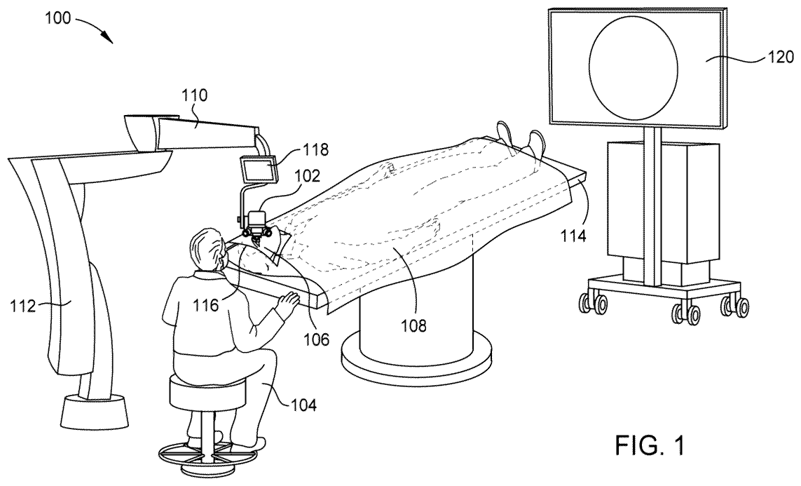

FIG. 1 illustrates an example system 100 in which an illuminated ophthalmic surgical instrument (hereinafter “the illuminated instrument”) may be used during ophthalmic surgeries, including glaucoma surgeries. The system 100 includes an ophthalmic microscope 102. A surgeon 104 uses the ophthalmic microscope 102 to visualize structures on and in an eye 106 of a medical patient 108 undergoing a surgery. The ophthalmic microscope 102 is supported on, in this illustration, an adjustable overhead arm 110 of a microscope support pedestal 112. The patient 108 may be supported on an operating table 114. The ophthalmic microscope 102 is movable with the overhead arm 110 in three dimensions so that the surgeon 104 can position the ophthalmic microscope 102 as desired with respect to the eye 106 of the patient 108.

In certain embodiments, the ophthalmic microscope 102 comprises a high resolution, high contrast stereo viewing surgical microscope. The ophthalmic microscope 102 will often include a monocular eyepiece 116 or binocular eyepieces 116, through which the surgeon 104 will have an optically magnified view of the relevant eye structures that the surgeon 104 will need to see to accomplish a given surgery or diagnose an eye condition of the patient 108.

The ophthalmic microscope 102 includes a digital camera and broadband light source for capturing color (red, green, and blue) images, a multi-spectral imaging (MSI) device, and/or other type of imaging device. Digital images captured using the camera may be displayed on a display device within the ophthalmic microscope 102.

The ophthalmic microscope 102 may include two display devices that are viewable through binocular eyepieces 116 and that display images of the patient's eye 106 captured from different viewpoints by two cameras to provide stereoscopic viewing. For example, the ophthalmic microscope 102 may be implemented as the NGENUITY 3D VISUALIZATION SYSTEM provided by Alcon Inc. of Fort Worth Texas.

Images from the ophthalmic microscope 102 may be additionally or alternatively be displayed on one or more display devices. For example, the one or more display devices may include a display device 118 fastened to the supporting arm 110 above the ophthalmic microscope 102.

In order to relieve the surgeon 104 from the need to constantly look into the eye pieces 116 to obtain a stereoscopic view, the one or more display devices may include a display device 120 that may be implemented as a three-dimensional display device. The display device 120 may therefore provide a stereoscopic view of images captured using the ophthalmic microscope 102. The display device 120 may be embodied as any type of three-dimensional display device known in the art, including those that do or do not use special filtering glasses. For some types of three-dimensional display devices, the perception of three dimensions requires that the distance of the viewer from the display device 120 be within a threshold distance from the display device. The display device 120 may be mounted to a cart, a manually adjustable or robotic arm, or other manually or automatically adjustable support.

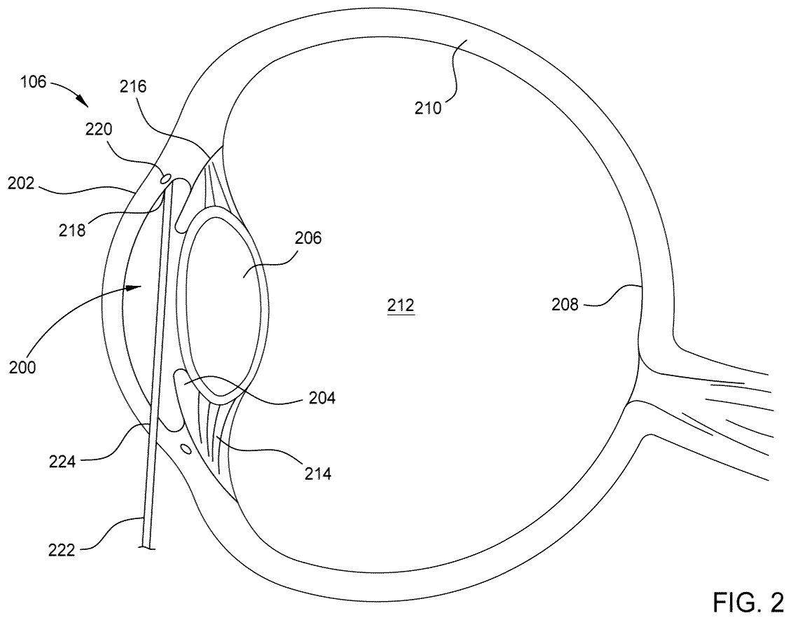

FIG. 2 is a diagram illustrating structures in and around anterior chamber 200 that are relevant to the treatment of glaucoma. The anterior chamber 200 of the eye is located behind the transparent and spherical cornea 202 through which light enters the eye. The iris 204 is a ring of muscles defining the pupil of the eye through which light passes. The crystalline lens 206 is located behind the pupil and, together with the cornea 202, focuses light onto the light sensitive cells of the retina 208. The retina 208 is formed on the interior of the globe 210 of the eye opposite the anterior chamber 200. The globe 210 of the eye between the lens 206 and the retina 208 is occupied by a transparent gel known as the vitreous 212.

The ciliary body 214 includes ligaments and muscles that connect the iris 204 and lens 206 to the choroid 216 of the eye. The muscles of the ciliary body 214 are responsible for altering the shape of the lens 206. The choroid 216 is a vascularized layer lining the globe 210 of the eye.

The ciliary body 214 produces the aqueous humor, which is the fluid that occupies the anterior chamber 200. The aqueous humor washes over the lens 206 and iris 204 and flows to the perimeter of the anterior chamber 200. The perimeter of the anterior chamber includes structures that, when functioning normally, allow the aqueous humor to drain. These structures include the trabecular meshwork 218 and Schlemm's canal 220. The trabecular meshwork 218 seems to act as a filter, limiting the outflow of aqueous humor and providing a back pressure that directly relates to IOP. Schlemm's canal 220 is located beyond the trabecular meshwork 218. Schlemm's canal 220 is fluidically coupled to collector channels (not shown) allowing aqueous humor to flow out of the anterior chamber 200.

Glaucoma may be treated by inserting the illustrated rod 222 into the anterior chamber 200, such as through an incision in the limbus 224 at the boundary between the cornea 202 and the sclera (white) of the eye. The rod 222 is then used to place an incision and possibly a shunt in one or more structures at the perimeter of the anterior chamber 200 to facilitate drainage of the aqueous humor. For example, an incision or shunt may be placed in the trabecular meshwork 218 to facilitate drainage into Schlemm's canal 220. In other approaches, a shunt extends from the anterior chamber into a suprachoroidal space between the choroid 216 and globe 210 of the eye.

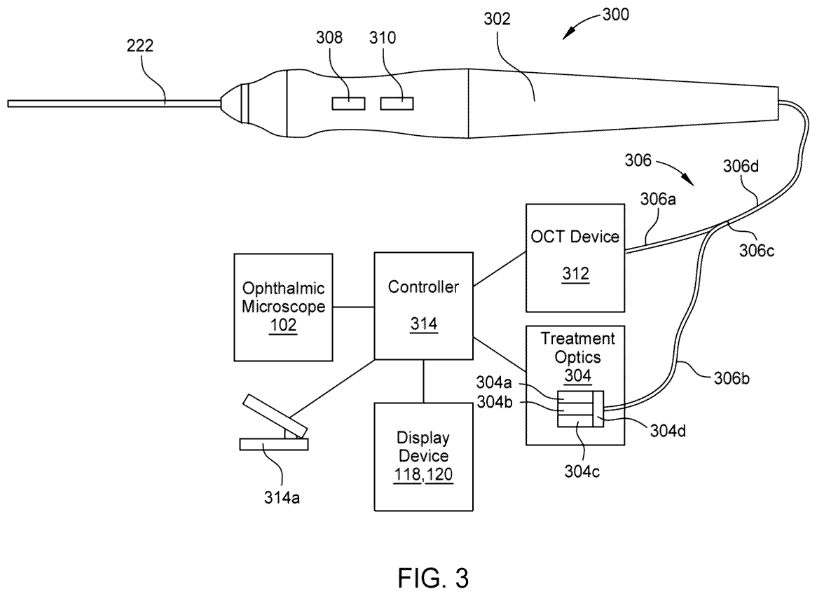

Referring to FIG. 3, a surgical instrument 300 may include the rod 222 and a handpiece 302 to which the rod 222 is mounted. The rod 222 is coupled through the handpiece 302 to treatment optics 304, such as by an optical fiber 306. The treatment optics 304 may be external to the handpiece 302 or mounted within the handpiece 302. The handpiece 302 is designed to be held in the hand of a surgeon 104 and may have one or more buttons 308, 310 or other interface elements mounted thereto and electrically coupled to the treatment optics 304 to control operation thereof.

The treatment optics 304 may include one or both of a treatment light source 304a and an illumination light source 304b. The treatment light source 304a may be embodied as a laser providing treatment light of sufficient intensity to disintegrate tissue up to a prescribed distance from the tip of the rod 222. For example, the treatment light source 304a may be embodied as an infrared laser, such as an infrared laser diode. Parameters of the treatment light source 304a that may be controlled may include pulse energy, pulse duration, pulse frequency, and a number of pulses emitted (e.g., per pressing of a button 308, 310). For example, the treatment light source 304a may emit pulses with between 0.75 and 1.25 millijoules per pulse at a rate of 0.75 to 1.25 kHz. For example, the treatment light source 304a may emit pulses with a pulse energy of 1 millijoule at a rate of 1 kHz. The number of pulses required to make an incision in the trabecular meshwork 218 may be, for example, from 50 to 60 pulses.

The illumination light source 304b may emit light in the visible wavelength range (e.g., 380 to 700 nm). The illumination light source 304b may be implemented as one or more light emitting diodes (LED) emitting broadband light or a set of LEDs each with a peak intensity wavelength at a different point in the visible spectrum, such as red, green, blue, or other colors. Parameters of the illumination light source 304b that may be controlled may include intensity, pulse frequency (e.g., flashing frequency), pulse duration, color (e.g., different combinations of intensities for two or more LEDs having two or more different peak intensity wavelengths).

In some embodiments, the treatment optics 304 may be used for coordinating operation of the OCT device 312 with the illumination light source 304a. For example, the illumination light source 304a may be turned off while measurements are being performed using the OCT device 312 and turned back on when measurements are completed.

The treatment optics 304 may include a camera 304c capable of capturing images using light transmitted through an optical fiber. For example, the camera 304c may be a borescope.

The treatment optics 304 may include combining optics 304d configure to input light from the treatment and illumination light sources 304a, 304b to the optical fiber 306, such as by means of a beam splitter or other combining optics. The combining optics 304d may transmit light reflected into the optical fiber 306 to the camera 304c. In some embodiments, treatment and illumination light sources 304a, 304b may transmit light through different cores of the optical fiber 306, such as through different cores of a multi-core optical fiber. Likewise, the camera 304c may receive light using a different core than the treatment and illumination light sources 304a, 304b.

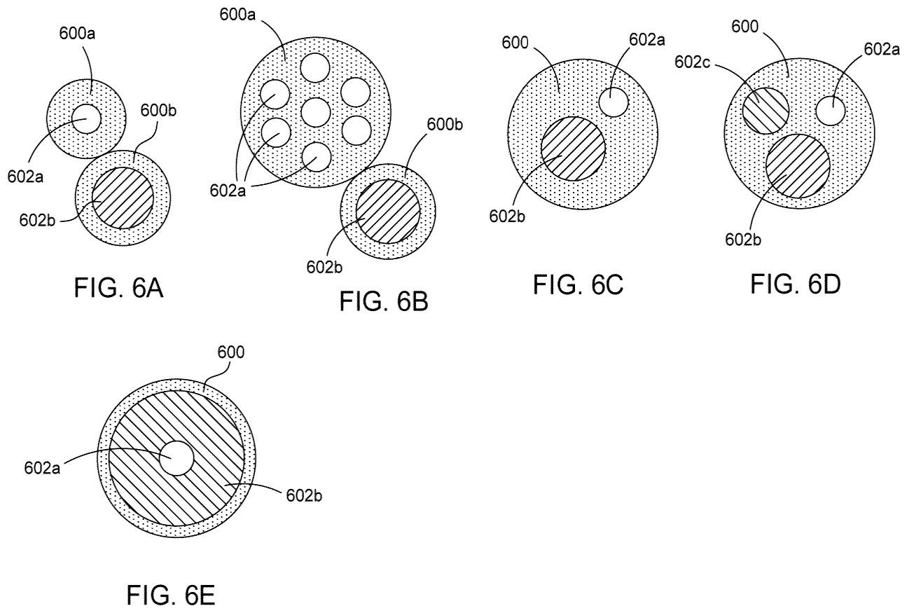

An optical coherence tomography (OCT) device 312 may be coupled to the optical fiber 306. The OCT device 312 may be a fiber OCT according to any approach known in the art. The OCT device 312 may be used without corresponding scanning optics and may therefore be considered to be a one-dimensional OCT device 312, though two-dimensional options are possible (see FIGS. 6A to 6E and corresponding description). In particular, the OCT device 312 may transmit light from an OCT light source (e.g., laser) through the optical fiber 306 and receive reflected light that is reflected back into the optical fiber 306. The OCT device 312 may then evaluate the reflected light to determine the transparency of structures that reflected the reflected light. For example, the OCT device 312 may perform time-domain or spectral domain evaluation of the reflected light with respect to a reference beam from the OCT light source.

The optical fiber 306 may provide a combined optical channel for light from the treatment optics 304 and light traveling to and from the OCT device 312. For example, the optical fiber 306 may include a first portion 306a coupled to the OCT device 312, a second portion 306b coupled to the treatment optics 304 and a junction 306c that guides light from the first portion 306a and second portion 306b into common portion 306d that is coupled to the surgical instrument 300. The common portion 306d may extend to a distal end of the rod 222 or may be coupled to another segment of optical fiber that extends to the distal end of the rod 222. The common portion 306d may conduct light from the first portion 306a and second portion 306b in a common core or may include separate cores, each core conducting light from only one of the first portion 306a and the second portion 306b.

The junction 306c further guides at least a portion of light reflected back into the common portion 306d into the first portion 306a, which conducts the reflected light to the OCT device 312 for detection. The optical path length between the distal end of the rod 222 and the detector of the OCT device 312 may affect the operation of the OCT device 312. Accordingly, lengths of one or both of the first portion 306a and the common portion 306d may be selected to correspond to the optical path length needed for correct operation of the OCT device 312.

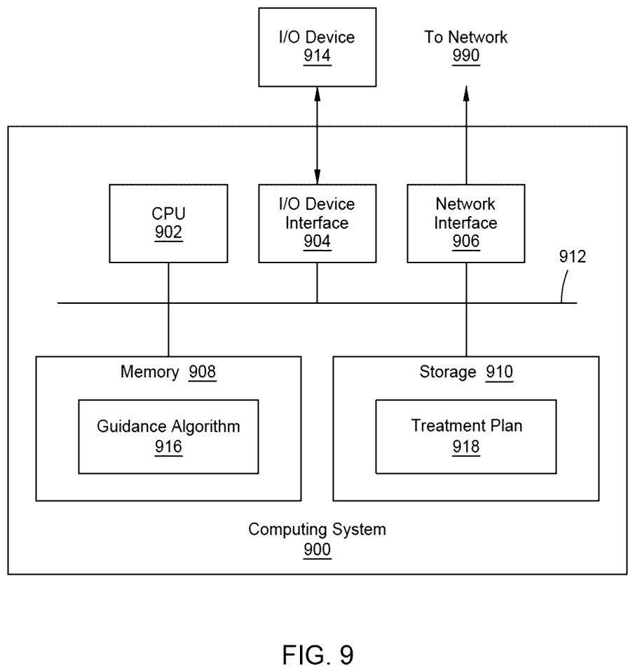

Operation of some or all of the ophthalmic microscope 102, treatment optics 304, and OCT device 312 may be controlled by a controller 314. The controller 314 may be implemented as a general-purpose computing device, such as by a computing system 900 as described below with respect to FIG. 9. The controller 314 may also be implemented as a dedicated circuit or device, such as an application specific integrated circuit (ASIC), field programable gate array, or other computing device.

The controller 314 may be coupled to a display device 120, 118 and/or a display device internal to the ophthalmic microscope 102. The controller 314 may, for example, control capture of imaging data by the OCT device 312, synchronizing output of the treatment optics 304 with image capture by the OCT device 312, output imaging data captured by the OCT device 312 on a display device 118, 120, and/or perform other functions. A surgeon 104 may provide inputs to the controller 314. For example, a foot pedal 314a or other input device may enable the surgeon 104 to provide inputs to the controller 314.

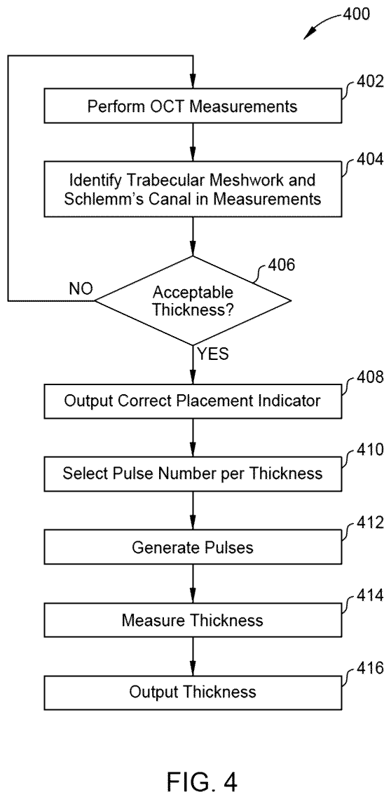

For example, referring to FIG. 4, the controller 314 may implement the illustrated method 400. The method 400 may be preceded by the surgeon 104 positioning the distal end of the rod 222 in contact with the trabecular meshwork 218 or within a threshold distance (e.g., within 0.5 mm). The method 400 may be invoked by the surgeon 104, such as by pressing a button 308, 310, pressing the foot pedal 314a, or providing some other input.

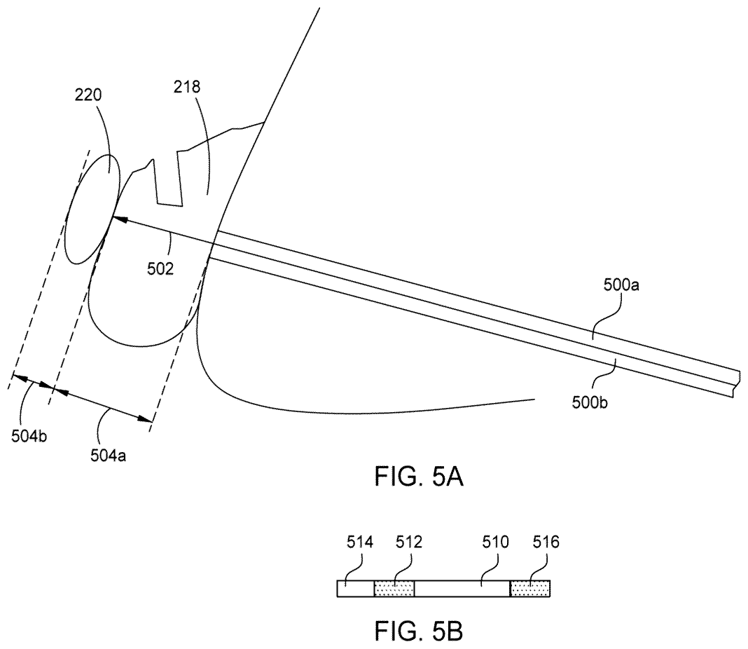

The method 400 may include performing, at step 402, OCT measurements using the OCT device 312 and identifying, at step 404, boundaries between the trabecular meshwork 218 and Schlemm's canal 220. An example approach for implementing steps 402 and 404 are illustrated by FIGS. 5A and 5B.

FIGS. 5A and 5B illustrate two optical channels 500a, 500b placed having distal ends thereof adjacent (e.g., within 0.5 mm) or contacting the trabecular meshwork 218. The optical channels 500a, 500b may extend within the rod 222. The optical channels 500a, 500b may be implemented as different cores of the same fiber or distinct optical fibers (see FIGS. 6A to 6E and corresponding description below). The optical channel 500a may conduct light to and from the OCT device 312 and the optical channel 500b may conduct light from the treatment optics 304. In particular, optical channel 500b may transmit light from a treatment light source 304a.

Light 502 emitted from the optical channel 500a is transmitted through, and at least partially scattered by, the trabecular meshwork 218. A portion of the light 502 reaches Schlemm's canal 220. The fluid within Schlemm's canal 220 will typically not scatter the light 502 as much as the trabecular meshwork 218. A portion of the light 502 that passes through Schlemm's canal 220 may be scattered and/or transmitted by tissue of the trabecular meshwork 218 on the far side of Schlemm's canal 220 relative to the optical channel 500a.

The light scattered by the trabecular meshwork 218 and the fluid within Schlemm's canal 220 is detected by the OCT device 312 resulting in a one-dimensional array of intensity values as shown schematically in FIG. 5B. For example, region 510 of the array may correspond to the portion of the trabecular meshwork 218 between the optical channel 500a and Schlemm's canal 220, region 512 of the array may correspond to the fluid within Schlemm's canal, and region 514 of the array may correspond to tissue on the far side of Schlemm's canal. The boundaries between the regions 510, 512, 514 may be determined by identifying changes in intensity corresponding to transitions between the tissue of the trabecular meshwork 218 and the transparent fluid within Schlemm's canal, that is relatively more transparent than the tissue of the trabecular meshwork 218. For example, a first portion of the array below a threshold intensity may be deemed to be the region 512 with portions on either side of the first portion being deemed to be the regions 510 and 514.

The aqueous humor of the anterior chamber 200 between the distal end of the optical channel 500a and the trabecular meshwork 218 may also be apparent in the array of reflectance values due to the reduced scattering of the aqueous humor compared to the trabecular meshwork 218. For example, region 516 may indicate the extent of the aqueous humor between the distal end of the optical channel 500a and the trabecular meshwork 218. Region 516 may be absent when the distal end of the optical channel 500a is in contact with the trabecular meshwork 218.

Referring again to FIG. 4, while continuing to refer to FIGS. 5A and 5B, the method 400 may include evaluating, at step 406, the thickness of the trabecular meshwork 218 and possibly a depth of Schlemm's canal 220 as represented in the measurements from step 402.

For example, the extent (e.g., number of elements in the array) of the region 510, may correspond to a distance 504a between the outer surface of the trabecular meshwork 218 (e.g., facing into the anterior chamber and interfacing with the aqueous humor therein) and an inner surface of Schlemm's canal 220. The distance 504b may correspond to the extent of the region 512. The distance 504a and the distance 504b may both be measured along the optical axis defined by the optical channel 500a, e.g., the optical axis of the light 502. The optical axis of the light 502 may be defined as the central axis of a Gaussian beam emitted from the optical channel 500a. The optical axis may be the same as the central axis of the optical channel 500a or different therefrom according to a cleavage angle at a face of the optical channel 500a (see, e.g., 7A, 7B, 7C, and 7D and corresponding description).

The distance 504a and distance 504b may be used to determine whether the optical channels 500a, 500b are positioned correctly relative to Schlemm's canal 220. For example, an incision to facilitate drainage to Schlemm's canal 220 may ideally be placed where the distance 504a is at a minimum and possibly where the distance 504b is at a maximum. A first range of acceptable values for the distance 504a and/or a second range of acceptable values for the distance 504b may be predefined. For example, the first and second range may be obtained by measuring these values using one or more pre-operative (e.g., prior to insertion of the rod 222) images of the eye 106, such as one or more two-dimensional or three-dimensional OCT images of the eye 106. For example, a surgeon or other human operator may generate one or more incision definitions, each incision definition including a location on a surface of the trabecular meshwork 218 facing the anterior chamber and a direction. The location and direction may be selected to increase drainage. For example, the locations may be distributed radially about the optical axis of the eye 106.

For a given radial position, the location and direction may be selected to be approximately (e.g., within 0.1 mm) through a thinnest portion of the trabecular meshwork 218. Each incision definition may have a corresponding thickness of the trabecular meshwork and thickness of Schlemm's canal at the location and along the direction of the incision definition. The first and second ranges for an incision definition may therefore be defined as an acceptable variation (e.g., within 0.1 mm) from the thickness of the trabecular meshwork and thickness of Schlemm's canal for the incision definition.

Radial positioning of the distal end of the rod 222 may be facilitated using the ophthalmic microscope 102: the surgeon may view the distal end of the rod 222 using the ophthalmic microscope 102 in order to guide positioning of the distal end of the rod 222. The controller 314 may superimpose guidance on images captured using the ophthalmic microscope 102, such as markings showing the radial position of each incision definition.

Step 406 may be performed without the benefit of pre-operative data, such as an incision definition as described above. For example, the surgeon 104 may move the distal end of the rod 222 relative to the trabecular meshwork 218 and a plurality of OCT measurements may be performed at a variety of positions and/or orientations of the distal end of the rod 222. The distances 504a and 504b may be obtained for each measurement. An acceptable distance 504a may then be calculated as the smallest of the distances 504a, or the smallest of the distances 504a plus a predefined tolerance.

If the distance 504a and possibly the distance 504b are not found, at step 406, to be acceptable (e.g., within first and second ranges), then steps 402-406 may be repeated. If the distance 504a and distance 504b are found to be acceptable, one or more additional steps may be performed. For example, the method 400 may include outputting, at step 408, an indicator indicating that the distal end of the optical channels 500a, 500b are correctly placed. The output may be displayed on one or more of the display devices 118, 120, in a display internal to the ophthalmic microscope, or other output device, such as a speaker outputting an audible tone or spoken message.

The method 400 may include selecting, at step 410, a pulse number according to the thickness of the trabecular meshwork 218, such as the length (e.g., number of array elements) in the region 510. Step 410 may include multiplying the length of the region 510 by a predefined conversion factor followed by rounding up or rounding down to an integer. Step 410 may include calculating the number of pulses as some other function of the length of the region 510. Step 410 may include looking up a number of pulses corresponding to the length of the region 510 in a lookup table. The pulse number may be a function of the pulse energy of a treatment light source 304a. Whichever approach is used to determine the number of pulses may be based on experiments with human or animal subjects relating the depth of disintegration to number of pulses. The number of pulses may also be selected to create an incision in the trabecular meshwork 218 on the far side of Schlemm's canal 220 when desired by a surgeon.

The method 400 may further include generating, at step 412, the number of pulses selected at step 410 and emitting the pulses into the trabecular meshwork 218 through the optical channel 500b. Step 412 may be performed automatically in response to step 406 or may be performed in response to an action of the surgeon 104, e.g., pressing a button 308, 310, pressing the foot pedal 314a coupled to the controller 314, or providing some other input.

The method 400 may include measuring, at step 414, the thickness of the trabecular meshwork 218, e.g., immediately following generating the pulses such that the optical axis of the rod 222 is substantially (e.g., within 0.001 mm and within 0.5 degrees of) the same orientation as when pulses were generated at step 412. Step 414 may be performed as described above with respect to step 402. The method 400 may include outputting, at step 416, a representation of the thickness. The representation may include a representation of a one-dimensional array of reflectance values (see, e.g., FIG. 5B) as measured at step 414, a numerical value representing the thickness, an indicator that the trabecular meshwork 218 is completely traversed, or other indicator. The representation of step 416 may be displayed on one or more of the display devices 118, 120, in a display internal to the ophthalmic microscope, or other output device, such as a speaker outputting an audible tone or spoken message.

For example, if a complete incision of the trabecular meshwork 218 between the anterior chamber and Schlemm's canal 220 is not complete, a surgeon 104 may assess drainage capability of the incomplete incision due to porosity of the trabecular meshwork 218. The surgeon 104 may then determine whether to make additional incisions if the drainage capability is insufficient.

In some embodiments, if the thickness measured at step 414 is non-zero, processing may repeat at step 410 with the calculation of a number of pulses for the remaining thickness followed by generating, at step 412, that number of pulses to complete the incision.

The method 400 is exemplary only and various modifications may be made. For example, steps 404 and 406 may be replaced with a human determination of whether a location and direction are acceptable. For example, a representation (see FIG. 5B and corresponding description) of the output of the OCT, distance 504a, and/or distance 504b may be output to the surgeon 104, such as on one or more of the display devices 118, 120, in a display internal to the ophthalmic microscope, or other output device, such as a speaker outputting an audible tone or spoken message. The surgeon may then select when to generate pulses at step 412.

The method 400 may be extended to further include evaluating the size of the region 516 corresponding to the extent of the aqueous humor interposed between the distal end of the optical channel 500a and the trabecular meshwork 218. An output to the surgeon 104 may be generated based on the size of the region 516. The output may be a numerical or graphical representation of the size of the region 516. The output may be a text, color, arrow, or other symbol serving as a direction indicator. For example, the direction indicator may communicate “too far” if the distal end of the optical channel 500a is not close enough to the trabecular meshwork 218. The direction indicator may communicate that a correct position has been achieved if the distal end of the optical channel 500a is deemed close enough to the trabecular meshwork 218 and not too close. The direction indicator may instruct the surgeon 104 to move away from the trabecular meshwork 218 if the distal end of the optical channel 500a is deemed too close to the trabecular meshwork 218.

The distal end of the optical channel 500a may be deemed close enough to the trabecular meshwork 218 if the size of the region 516 is below a first threshold distance, e.g., less than 0.1 millimeter. In some embodiments, a non-zero size for the region 516 may be desirable to ensure that the distal end of the optical channel 500a is not pressed too deeply into the trabecular meshwork 218. Accordingly, the distal end of the optical channel 500a may be deemed too close to the trabecular meshwork 218 if the size of the region 516 is below a second threshold distance that is smaller than the first threshold distance.

FIGS. 6A to 6E illustrate various configurations that may be used to implement the first optical channel 500a and the second optical channel 500b. The illustrated cross sections of optical fibers may be present within the rod 222 and possibly also in the common portion 306d.

Referring specifically to FIG. 6A, in a first example, the first optical channel 500a is implemented by first cladding 600a surrounding a first core 602a used to conduct light to and from the OCT device 312. The second optical channel 500b is implemented by second cladding 600b surrounding a second core 602b used to conduct light from the treatment optics 304. The first cladding 600a may be secured to the second cladding 600b by way of an adhesive, co-molding, or containment within a common tube or other outer layer. The combined thickness of the first cladding 600a and second cladding 600b may be sufficient to prevent substantial (e.g., greater than 1 dB) leakage of light between the first core 602a and the second core 602b.

Referring specifically to FIG. 6B, in a second example, the first optical channel 500a is implemented by first cladding 600a surrounding a plurality of cores 602a used to conduct detection light to and from the OCT device 312. The plurality of cores 602a are distributed spatially in two dimensions and may conduct light independently without substantial (e.g., greater than 1 dB) leakage of light therebetween. The OCT device 312 may independently perform measurements for each core 602a. The output of the OCT device 312 may therefore be a plurality of one-dimensional arrays constituting a volumetric image. The volumetric image may then be displayed to the surgeon 104 on a display device 118, 120 or a display device internal to the ophthalmic microscope to guide a surgeon in selecting a location and direction to place an incision. Likewise, the volumetric image may be processed by the controller 314 to identify a thinnest point in the trabecular meshwork and select a location and/or direction to place an incision for use according to steps 404 and 406 as described above with respect to FIG. 4. For the embodiment of FIG. 6B, step 414 may include locating the incision within the volumetric image (e.g., by identifying the aqueous humor within the volumetric image) and determining a depth of the incision within the volumetric image.

In the embodiment of FIG. 6B, the second optical channel 500b may likewise be implemented by second cladding 600b surrounding a second core 602b. The first cladding 600a may be secured to the second cladding 600b by way of an adhesive, co-molding, or containment within a common tube or other outer layer. The combined thickness of the first cladding 600a extending outwardly from the outermost cores 602a and second cladding 600b may be sufficient to prevent substantial (e.g., greater than 1 dB) leakage of light between the plurality of first cores 602a and the second core 602b.

Referring to FIG. 6C, the first optical channel 500a may be implemented by a first core 602a and the second optical channel 500b may be implemented by a second core 602b, the first core 602a and the second core 602b being contained within common cladding 600. A portion of the cladding 600 may extend between the first core 602a and the second core 602b to prevent substantial (e.g., greater than 1 dB) leakage of light between the first core 602a and the second core 602b. The embodiment of FIG. 6C may likewise include multiple first cores 602a that are distributed and used as for the embodiment of FIG. 6B.

Referring to FIG. 6D, the first optical channel 500a may be implemented by a first core 602a and the second optical channel 500b may be implemented by a second core 602b, the first core 602a and the second core 602b being contained within common cladding 600. A third core 602c may be embedded in the cladding 600. The additional core 602c may be used for one or more purposes. For example, the core 602c may be used to implement a fiberscope, which may conduct light to the camera 304c, such as a borescope. The borescope may be used to obtain images of the trabecular meshwork 218, and possibly any incision made therein. The output of the borescope may be displayed on a display device 118, 120 or a display device internal to the ophthalmic microscope 102.

A portion of the cladding 600 may extend among the first core 602a, the second core 602b, and the third core 602c to prevent substantial (e.g., greater than 1 dB) leakage of light between the first core 602a, the second core 602b, and the third core 602c. The embodiment of FIG. 6D may likewise include multiple first cores 602a that are distributed and used similar to the embodiment of FIG. 6B.

In the embodiment of FIG. 6E, the first optical channel 500a may be implemented by a first core 602a implemented as a single mode fiber. The second optical channel 500b may be implemented by a second core 602b extending around the first core 602a, e.g., concentric with the first core 602a. The second core 602b may be a mid-infrared (MIR) optical fiber. Crosstalk between the cores 602a, 602b may occur such that the light sources 304a, 304b may be turned off when the OCT 312 is being used to obtain measurements in order to avoid interference.

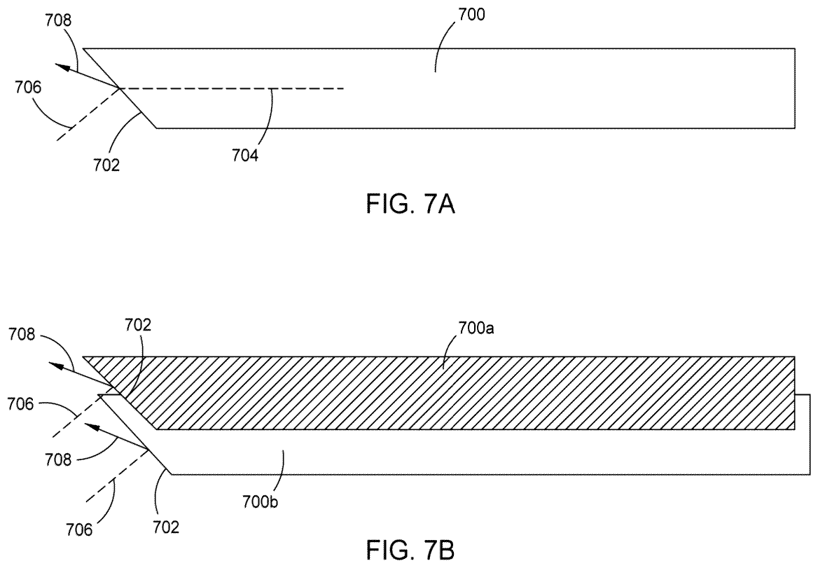

FIG. 7A illustrates an example implementation of an optical fiber 700 that may be used to implement the optical channel 500a, optical channel 500b, or any of the optical fiber configurations of FIGS. 6A to 6D. The optical fiber 700 may have a cleavage surface 702 that is non-perpendicular relative to a central axis 704 of the optical fiber 700 (e.g., a core of the optical fiber 700). For example, the normal vector 706 of the cleavage surface may define an angle of between 4 and 20 degrees relative to the central axis 704 of the optical fiber 700 intersecting the cleavage surface 702.

The non-perpendicular cleavage surface 702 may cause light 708 exiting the cleavage surface 702 to be non-parallel to the central axis 704 due to refraction upon exiting the cleavage surface 702, such as an angle of between 4 and 20 degrees. The non-parallel angle of the light 708 may facilitate imaging and/or incision placement relative to the trabecular meshwork 218 and Schlemm's canal 220. In particular, the geometry of the anterior chamber and the placement of the incision in the limbus 224 may limit options for the placement and orientation of the rod 222. Accordingly, the angle of the cleavage surface 702 may be selected to enable light 708 to be directed at an angle along which the rod 222 itself cannot be oriented.

The non-perpendicular cleavage surface 702 may additionally reduce back reflection of scattered light from the cleavage surface, thereby increasing the resolution of imaging data obtained with the OCT device 312 from light scattered by tissue of the patient.

Referring to FIG. 7B, multiple optical fibers 700a, 700b may be present each with a non-perpendicular cleavage surface 702 as described above. For example, one optical fiber 700a may implement the optical channel 500a and the other optical fiber 700b may implement the optical channel 500b. The cleavage surfaces 702 of the optical fibers 700a, 700b may be parallel to one another within manufacturing tolerances, such as by being cut in a same manufacturing step. The cleavage surfaces 702 may also be non-parallel to one another.

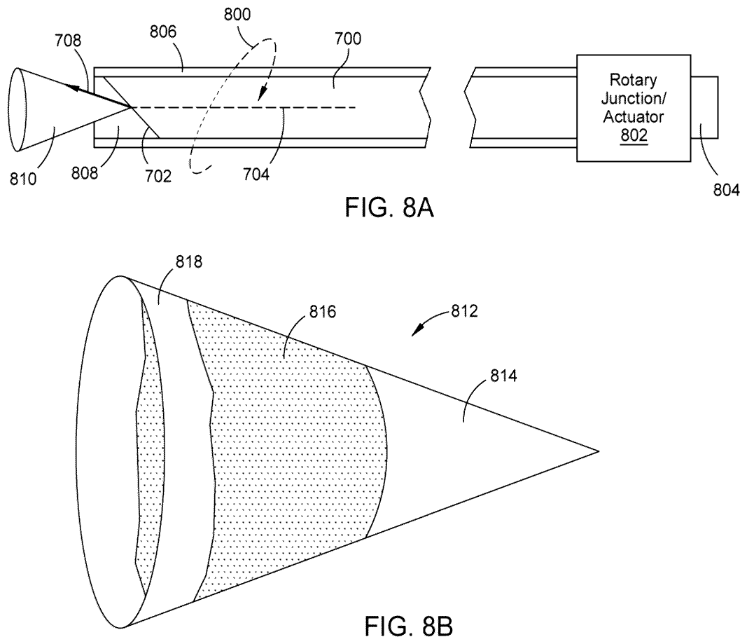

Referring to FIG. 8A, in some embodiments, an optical fiber 700 with a non-perpendicular cleavage surface 702 may be rotated during use, such as in rotational direction 800 about the central axis 704 of the optical fiber 700. The optical fiber 700 may be rotated using a rotary junction 802 that enables the transmission of light to and from a non-rotating optical fiber 804. The rotary junction 802 may further include a rotational actuator that rotates the optical fiber 700. The rotation of the optical fiber 700 may be rapid (e.g., one or more revolution per second) or relatively slow (e.g., less than one revolution per second). Actuation of the optical fiber 700 may be manually induced by a surgeon 104 or by a motor within the rotary junction 802. The non-rotating optical fiber 804 may be the common portion 306d or coupled thereto.

The optical fiber 700 may be positioned within a sheath 806 to prevent rubbing against tissue of the eye 106 during use. Likewise, a wedge 808 may be fitted over the cleavage surface to reduce stirring of fluid or damage to patient tissue by the cleavage surface 702 during rotation. The wedge 808 may have an index of refraction substantially (e.g., within 1 percent) the index of refraction of the aqueous humor.

Rotation of the optical fiber 700 causes the direction of the light 708 to rotate about a surface of a cone 810. Accordingly, the output of the OCT device 312 is a series of one-dimensional arrays that each correspond to a different angular position around the cone 810.

For example, FIG. 8B illustrates an example representation of the output of the OCT device 312 using the embodiment of FIG. 8A. The one-dimensional arrays may be represented as a cone 812 showing the angular position at which each one-dimensional array was obtained. The cone 812 will therefore include regions 814, 816, 818 corresponding to the trabecular meshwork 218 between the anterior chamber and Schlemm's canal, Schlemm's canal, and the portion of the trabecular meshwork 218 on the far side of Schlemm's canal, respectively.

A rendering of the cone 812 may then be displayed to the surgeon 104 on a display device 118, 120 or a display device internal to the ophthalmic microscope to guide a surgeon in selecting a location and direction to place an incision. Likewise, the cone 812 may be processed by the controller 314 to identify a thinnest point in the trabecular meshwork and select a location and/or direction to place an incision for use according to steps 404 and 406 as described above with respect to FIG. 4.

FIG. 9 illustrates an example computing system 900. The controller 314 may have some or all of the attributes of the computing system 900. The ophthalmic microscope 102 and the display devices 118, 120 may likewise incorporate a computing device having some or all of the attributes of the computing system 900.

As shown, computing system 900 includes a central processing unit (CPU) 902, one or more I/O device interfaces 904, which may allow for the connection of various I/O devices 914 (e.g., keyboards, displays, mouse devices, pen input, etc.) to computing system 900, network interface 906 through which computing system 900 is connected to network 990, a memory 908, storage 910, and an interconnect 912.

CPU 902 may retrieve and execute programming instructions stored in the memory 908. Similarly, CPU 902 may retrieve and store application data residing in the memory 908. The interconnect 912 transmits programming instructions and application data, among CPU 902, I/O device interface 904, network interface 906, memory 908, and storage 910. CPU 902 is included to be representative of a single CPU, multiple CPUs, a single CPU having multiple processing cores, and the like.

Memory 908 is representative of a volatile memory, such as a random access memory, and/or a nonvolatile memory, such as nonvolatile random access memory, phase change random access memory, or the like. As shown, memory 908 may store executable code implementing a guidance algorithm 916 defining executable code, machine learning models, or other instructions for performing the method 400.

Storage 910 may be non-volatile memory, such as a disk drive, solid state drive, or a collection of storage devices distributed across multiple storage systems. Storage 910 may optionally store a treatment plan 918. The treatment plan may include one or more labeled reference images to facilitate detection of anatomy within the eye 106, one or more incision definitions, parameters for activating the treatment light source 304a (e.g., pulse energy, pulse duration), or other information to facilitate administration of glaucoma treatments.

### Additional Considerations

The preceding description is provided to enable any person skilled in the art to practice the various embodiments described herein. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

The various illustrative logical blocks, modules and circuits described in connection with the present disclosure may be implemented or performed with a general purpose processor, a digital signal processor (DSP), an application specific integrated circuit (ASIC), a field programmable gate array (FPGA) or other programmable logic device (PLD), discrete gate or transistor logic, discrete hardware components, or any combination thereof designed to perform the functions described herein. A general-purpose processor may be a microprocessor, but in the alternative, the processor may be any commercially available processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

A processing system may be implemented with a bus architecture. The bus may include any number of interconnecting buses and bridges depending on the specific application of the processing system and the overall design constraints. The bus may link together various circuits including a processor, machine-readable media, and input/output devices, among others. A user interface (e.g., keypad, display, mouse, joystick, etc.) may also be connected to the bus. The bus may also link various other circuits such as timing sources, peripherals, voltage regulators, power management circuits, and the like, which are well known in the art, and therefore, will not be described any further. The processor may be implemented with one or more general-purpose and/or special-purpose processors. Examples include microprocessors, microcontrollers, DSP processors, and other circuitry that can execute software. Those skilled in the art will recognize how best to implement the described functionality for the processing system depending on the particular application and the overall design constraints imposed on the overall system.

If implemented in software, the functions may be stored or transmitted over as one or more instructions or code on a computer-readable medium. Software shall be construed broadly to mean instructions, data, or any combination thereof, whether referred to as software, firmware, middleware, microcode, hardware description language, or otherwise. Computer-readable media include both computer storage media and communication media, such as any medium that facilitates transfer of a computer program from one place to another. The processor may be responsible for managing the bus and general processing, including the execution of software modules stored on the computer-readable storage media. A computer-readable storage medium may be coupled to a processor such that the processor can read information from, and write information to, the storage medium. In the alternative, the storage medium may be integral to the processor. By way of example, the computer-readable media may include a transmission line, a carrier wave modulated by data, and/or a computer readable storage medium with instructions stored thereon separate from the wireless node, all of which may be accessed by the processor through the bus interface. Alternatively, or in addition, the computer-readable media, or any portion thereof, may be integrated into the processor, such as the case may be with cache and/or general register files. Examples of machine-readable storage media may include, by way of example, RAM (Random Access Memory), flash memory, ROM (Read Only Memory), PROM (Programmable Read-Only Memory), EPROM (Erasable Programmable Read-Only Memory), EEPROM (Electrically Erasable Programmable Read-Only Memory), registers, magnetic disks, optical disks, hard drives, or any other suitable storage medium, or any combination thereof. The machine-readable media may be embodied in a computer-program product.

A software module may comprise a single instruction, or many instructions, and may be distributed over several different code segments, among different programs, and across multiple storage media. The computer-readable media may comprise a number of software modules. The software modules include instructions that, when executed by an apparatus such as a processor, cause the processing system to perform various functions. The software modules may include a transmission module and a receiving module. Each software module may reside in a single storage device or be distributed across multiple storage devices. By way of example, a software module may be loaded into RAM from a hard drive when a triggering event occurs. During execution of the software module, the processor may load some of the instructions into cache to increase access speed. One or more cache lines may then be loaded into a general register file for execution by the processor. When referring to the functionality of a software module, it will be understood that such functionality is implemented by the processor when executing instructions from that software module.

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112 (f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

What is claimed is:

1. A system comprising:
a light source configured to emit treatment light in order to alter patient tissue;
an optical coherence tomography (OCT) device;
a surgical instrument including a distal portion configured to be inserted into an eye of a patient;
an optical fiber coupled to the light source and to the OCT device, the optical fiber configured to (a) conduct the treatment light along the distal portion to be emitted from a tip of the distal portion and (b) conduct detection light from the OCT device along the distal portion to be emitted from the tip of the distal portion and conduct a reflected portion of the detection light received at the tip of the distal portion to the OCT device; and
a controller coupled to the OCT device and the light source, the controller configured to:
obtain image data from the OCT device; and
operate the light source according to the image data.

2. The system of claim 1, wherein the controller is configured to operate the light source according to the image data by:
evaluating a thickness of a trabecular meshwork of the eye of the patient according to the image data;
selecting a pulse number according to the thickness; and
emit the pulse number of pulses from the light source.

3. The system of claim 2, wherein the light source is a laser.

4. The system of claim 1, wherein the controller is further configured to output guidance according to the image data.

5. The system of claim 4, wherein the controller is configured to:
evaluate a thickness of a trabecular meshwork of the eye of the patient between the tip of the distal portion and Schlemm's canal of the eye of the patient according to the image data; and
output the guidance according to the thickness.

6. The system of claim 1, wherein the optical fiber includes first cladding including a first core and second cladding including a second core, the first core configured to perform (a) and the second core configured to perform (b).

7. The system of claim 1, wherein the optical fiber includes cladding having a first core and a second core embedded therein, the first core configured to perform (a) and the second core configured to perform (b).

8. The system of claim 1, wherein:
the optical fiber includes a first core configured to perform (a) and a plurality of second cores configured to perform (b); and
the image data is a volumetric image.

9. The system of claim 1, wherein the distal portion defines a central axis, the tip of the distal portion configured to emit at least one of the treatment light and the detection light at a non-parallel angle of at least 4 degrees relative to the central axis.

10. The system of claim 9, further comprising a rotational actuator configured to rotate the optical fiber.

11. A method comprising:
inserting a distal portion of a surgical instrument into an eye of a patient, the distal portion including an optical fiber;
transmitting first light from an optical coherence tomography (OCT) device through the optical fiber and emitting the first light from the optical fiber onto tissue of the eye of the patient;
detecting, by the OCT device, a reflected portion of the first light to obtain image data; and
guiding, by a controller, operation of a light source according to the image data, the light source transmitting second light through the optical fiber onto tissue of the eye of the patient to alter the tissue.

12. The method of claim 11, wherein the tissue is a trabecular meshwork of the eye.

13. The method of claim 12, wherein guiding operation of the light source comprises:
evaluating, by the controller, a thickness of the trabecular meshwork according to the image data;
selecting, by the controller, a pulse number according to the thickness; and
emitting, by the controller, the pulse number of pulses from the light source.

14. The method of claim 12, wherein guiding operation of the light source comprises:
evaluating, by the controller, a thickness of a trabecular meshwork of the eye of the patient between a tip of the distal portion and Schlemm's canal of the eye of the patient according to the image data; and
outputting, by the controller, guidance to an output device according to the thickness.

15. The method of claim 11, wherein the optical fiber includes first cladding including a first core and second cladding including a second core, the first core transmitting the first light and the second core transmitting the second light.

16. The method of claim 11, wherein the optical fiber includes cladding having a first core and a second core embedded therein, the first core transmitting the first light and the second core transmitting the second light.

17. The method of claim 11, wherein the optical fiber includes a plurality of first cores transmitting the first light and a second core transmitting the second light.

18. The method of claim 17, wherein the image data is a volumetric image.

19. The method of claim 11, wherein the distal portion defines a central axis, a tip of the distal portion emitting at least one of the first light and the second light at a non-parallel angle of at least 4 degrees relative to the central axis.

20. The method of claim 19, further rotating the optical fiber.
